(19) United States
(12) Patent Application Publication
(10) Pub. No.: US 2026/0224445 A1
(43) Pub. Date: Aug. 6, 2026

(54) DENTAL VARNISH

(71) Applicant: Toothshine Limited, London (GB)

(72) Inventors: Sarah HAFFNER, London (GB); Michael HAFFNER, London (GB); Kamil Bugra TOGA, London (GB)

(73) Assignee: Toothshine Limited, London (GB)

(21) Appl. No.: 19/154,884

(22) PCT Filed: Feb. 8, 2024
(86) PCT No.: PCT/GB2024/050349

§ 371 Date: Aug. 8, 2025

(30) Foreign Application Priority Data
Feb. 10, 2023 (GB) 2301957.3

(51) Int. Cl. A61K 6/20; A61K 6/62; A61K 6/77; A61K 6/78; A61K 6/887

(52) U.S. Cl. CPC A61K 6/20; A61K 6/62; A61K 6/77; A61K 6/78; A61K 6/887

(57) ABSTRACT

The present invention relates to a dental varnish composition including a resin composition which includes an adhesive resin. The adhesive resin includes the acidic monomer 10-methacryloyloxydecyl dihydrogen phosphate (MDP); a base resin comprising the high molecular weight monomer bisphenol A glycidyl methacrylate (Bis-GMA); a solvent including one or more alcohols, water and optionally propylene glycol in an amount of from about 1 to about 5 wt % of the varnish composition; a photoinitiator; and a macromolecular emulsifier consisting of polyvinyl pyrolidone (PVP). The resin composition also includes a pigment composition which includes 2-hydroxyethylmethacrylate (HEMA) or salts thereof, pigment, and optionally a filler.

The present invention relates to a dental varnish. The dental varnish is semi-permanent, and can be removed and reapplied. The dental varnish changes the colour and appearance of teeth. The present invention also relates to a cosmetic method of whitening, colouring and/or changing the appearance of teeth.

Methods of whitening or colouring teeth to improve or change their appearance are well-known. However, such methods can be complex, involving multiple visits to a dental professional and the use of potentially toxic chemicals such as bleaching agents. Furthermore the resulting colour is unpredictable. These treatments can also lead to a permanent change in the appearance of teeth. In some cases it may be desirable for the change in colour or appearance of teeth to only last for a short period of time, for example for a special occasion, and/or for the dental varnish composition to be capable of being removed and reapplied, in much the same way as nail varnish. Home treatments for whitening teeth are also known, but these can be less effective and/or difficult to use.

Known whitening techniques also may not address other imperfections of teeth, such as surface imperfections.

US20190269579A1 disclosed a dental varnish suitable for changing the colour and appearance of teeth. The formulations disclosed herein are “shake-before-use” formulations which require agitation to form a homogeneous composition before use.

However, there is a need to provide improved dental varnish compositions that change the colour or appearance of teeth in a predictable manner. In particular, there is a need for a semi-permanent dental varnish that changes the colour and appearance of teeth on a semi-permanent basis, and is stable and resistant to phase separation. There is also a need for a cosmetic method of whitening, colouring and/or changing the appearance of teeth utilising such a composition.

### STATEMENT OF INVENTION

Accordingly, the present invention provides a dental varnish composition comprising:
a) a resin composition comprising:
   i) an adhesive resin comprising the acidic monomer 10-methacryloyloxydecyl dihydrogen phosphate (MDP) in an amount of from about 10 to about 30 wt % of the varnish composition;
   ii) a base resin comprising the high molecular weight monomer bisphenol A glycidyl methacrylate (Bis-GMA) in an amount of from about 15 to about 30 wt % of the varnish composition;
   iii) a solvent comprising:
      one or more alcohols in an amount of from about 15 to about 35 wt % of the varnish composition;
      water in an amount of from about 10 to about 25 wt % of the varnish composition; and
      optionally propylene glycol in an amount of from about 1 to about 5 wt % of the varnish composition;
   iv) a photoinitiator in an amount of from about 0.1 to about 2 wt % of the varnish composition;
   v) a macromolecular emulsifier consisting of polyvinyl pyrolidone (PVP) in an amount of from about 1 to about 10 wt % of the varnish composition; and
b) a pigment composition comprising:
   i) 2-hydroxyethylmethacrylate (HEMA) or salts thereof in an amount of from 1 to 10 wt % of the varnish composition;
   ii) pigment in an amount of from about 0.1 to about 15 wt % of the varnish composition; and
   iii) a filler in an amount of from about 1 to about 20 wt % of the varnish composition.

In a further aspect, the invention provides a dental varnish composition comprising:
a) a resin composition comprising:
   i) an adhesive resin comprising the acidic monomer 10-methacryloyloxydecyl dihydrogen phosphate (MDP) in an amount of from about 10 to about 30 wt % of the varnish composition;
   ii) a base resin comprising the high molecular weight monomer bisphenol A glycidyl methacrylate (Bis-GMA) in an amount of from about 15 to about 30 wt % of the varnish composition;
   iii) a solvent comprising:
      acetone in an amount of from about 15 to about 35 wt % of the varnish composition;
      water in an amount of from about 10 to about 25 wt % of the varnish composition; and
      optionally propylene glycol in an amount of from about 1 to about 5 wt % of the varnish composition;
   iv) a photoinitiator in an amount of from about 0.1 to about 2 wt % of the varnish composition;
   v) a macromolecular emulsifier consisting of polyvinyl pyrolidone (PVP) in an amount of from about 1 to about 10 wt % of the varnish composition; and
b) a pigment composition comprising:
   i) 2-hydroxyethylmethacrylate (HEMA) or salts thereof in an amount of from 1 to 10 wt % of the varnish composition;
   ii) pigment in an amount of from about 0.1 to about 15 wt % of the varnish composition; and
   iii) optionally a filler in an amount of from about 1 to about 20 wt % of the varnish composition.

Many of the components of these compositions are known for use as restorative materials in dentistry, in particular to fill dental cavities. However, the composition of the invention is not known for use as a varnish on the surface of teeth to change the colour or appearance of teeth. It has been surprisingly found that the above composition can be used to prepare a varnish, typically a semi-permanent varnish for teeth to improve or change the colour or cosmetic appearance of teeth. A great advantage of the composition of the invention is that it can be applied to teeth, removed and reapplied to teeth on a regular basis to predictably change the appearance of teeth; in much the same way as nail varnish can be used to change the appearance of nails. Compositions that can be applied to the surface of teeth to change the appearance of teeth are known; however these compositions cannot be easily removed and reapplied in contrast to the varnish compositions of the present invention. A particularly advantageous feature of the present invention is that the composition can provide a glossy and level surface of the tooth. The compositions of the invention can be “shake-before-use” formulations which require agitation to form a homogeneous composition before use. However, in preferred embodiments the compositions are stable and do not undergo phase separation, therefore not requiring shaking before use.

Typically, the MDP is present in an amount of from about 10 to about 25 wt %, optionally about 15 to about 25 wt %, optionally about 18 to about 22 wt %, optionally about 20 wt % of the varnish composition.

Preferably, the Bis-GMA is present in an amount of from about 15 to about 25 wt %, optionally about 15 to about 20 wt %, optionally about 20 to about 25 wt %, optionally about 16 to about 22 wt %, and optionally about 20 wt % of the varnish composition.

Typically, the one or more alcohols are present in an amount of from about 15 to about 30 wt %, optionally about 15 to about 25 wt %, optionally about 20 to about 25 wt %, optionally about 24 to about 25 wt % of the varnish composition. In a preferred embodiment, the one or more alcohols comprises ethanol, 1-propanol, 2-propanol, 1-butanol, 2-butanol, 2-methyl-1-propanol or combinations thereof. Preferably, the one or more alcohols comprise or consist of ethanol.

Preferably, the water is present in an amount of from about 12.5 to about 22.5 wt %, optionally about 14 to about 21 wt % of the varnish composition.

Preferably, propylene glycol is present. Preferably, the propylene glycol is present in an amount of from about 2 to about 4 wt %, optionally about 3 wt % of the varnish composition.

In one embodiment, the photoinitiator is present in an amount of from about 0.2 to about 1 wt %, optionally about 0.3 to 0.5 wt %, optionally about 0.4 wt % of the varnish composition. Typically, the photoinitiator comprises camphoroquinone (CQ) in combination with a co-initiator, trimethylbenzoylphenyl phosphate (TPO), monacylphosphine oxide, bisacylphosphine oxide, or a benzoyl germanium derivative and the composition optionally further comprises a photosensitizer.

Typically, the PVP is present in an amount of from about 3 to 8 wt %, optionally about 4 to about 6 wt % of the varnish composition. In a preferred embodiment, the PVP has a weight average molecular weight of at least about 400,000 Da, optionally about 400,000 Da to about 3.5 M Da, optionally wherein the PVP comprises PVP-90K.

Preferably, the pigment composition is present in an amount of from about 10 to 16 wt %, optionally about 12 wt % of the varnish composition.

Preferably, the HEMA is present in an amount of from about 3 to about 9 wt %, optionally about 4 to about 8 wt % of the varnish composition.

Typically, the pigment comprises one or more of mica, titanium dioxide, barium sulfate, cristobalite, iron oxide and combinations thereof.

Preferably, the filler comprises fumed silica, silica, silicate glass, quartz, barium silicate, strontium silicate, barium borosilicate, borosilicate, lithium silicate, amorphous silica, ammoniated or deammoniated calcium phosphate, alumina, zirconia, tin oxide, or combinations thereof. In one embodiment the filler comprises silicate glass, optionally nanoglass.

In one embodiment the pigment composition comprises HEMA, Mica and/or titanium dioxide, and nanoglass.

In one embodiment the pigment composition comprises HEMA and titanium dioxide, wherein the ratio of HEMA to titanium dioxide is from 1:1 to 2:3 in the pigment composition.

In one embodiment the pigment composition comprises HEMA, Mica and nanoglass, where the weight ratio of these components is 8:3:3, respectively in the pigment composition.

In a further embodiment, the pigment composition comprises HEMA, TiO₂ and barium sulfate.

In some embodiments the adhesive resin additionally comprises the acidic monomer 4-methacryloyloxyethyl trimellitate anhydride (4-META) in an amount of from about 0.5 to about 5 wt %.

In a further aspect, the invention provides a cosmetic method of whitening, colouring and/or changing the appearance of teeth, the method comprising applying a dental varnish composition as defined in any preceding claim to the tooth enamel of a subject, and curing the varnish with light having a wavelength in the range of from 380 nm to 550 nm, optionally 400 to 410 nm.

In one embodiment, the tooth enamel is cleaned and dried prior to applying the dental varnish, optionally using dry alcohol and a lint cloth.

In one embodiment of the method, after curing the varnish any excess varnish composition is removed.

In a preferred embodiment of the method, a top coat composition is applied after the curing step, optionally wherein the top coat composition forms a transparent layer.

In one embodiment, the top coat composition comprises a resin which is soluble in ethanol, optionally nitrocellulose, cellulose acetate butyrate or polyvinyl butyrate.

In one embodiment, the top coat composition comprises a an acrylate resin formed by curing one or more monomers comprising from two to five vinyl reactive groups, optionally wherein the top coat comprises triethylene glycol dimethacrylate (TEGDMA), bisphenol A-glycidyl methacrylate (bisGMA), pentaerythritol tri-acrylate, or pentaerythritol tetra-acrylate or combinations thereof.

In one embodiment, the dental varnish composition and/or the top coat composition are applied with a brush, optionally a brush comprising bristles made from a polymer selected from polypropylene, polyethylene, polyamide, or polycarbonate, optionally wherein the brush has a plurality of bristles each having a length of between about 10 to about 15 mm, preferably about 11 to about 14 mm, and a diameter of between about 50 and 75 microns.

In one embodiment, the method further comprises the step of mechanically removing the varnish.

In a further aspect, provided is a process for preparing a dental varnish wherein:
a) the resin composition components a)i) to v) as defined in claim 1 are mixed;
b) the pigment composition components b)i) to iii) as defined in claim 1 are mixed;
c) then the resin composition mixture formed in step a) and the pigment composition mixture formed in step b) are mixed to provide the dental varnish composition;
wherein mixing of the pigment composition in step b) involves preparing the pigment composition as a paste comprising HEMA, and agitating the composition using a high shear homogenizer that operates at about 200 to about 3,000 rpm minimum rate of shear, a three-roll-mill, or a ball mill system.

### DETAILED DESCRIPTION

The present invention provides a dental varnish composition comprising:
a) a resin composition comprising:
   i) an adhesive resin comprising the acidic monomer 10-methacryloyloxydecyl dihydrogen phosphate (MDP) in an amount of from about 10 to about 30 wt % of the varnish composition;
   ii) a base resin comprising the high molecular weight monomer bisphenol A glycidyl methacrylate (Bis-GMA) in an amount of from about 15 to about 30 wt % of the varnish composition;
   iii) a solvent comprising:
      one or more alcohols in an amount of from about 15 to about 35 wt % of the varnish composition;
      water in an amount of from about 10 to about 25 wt % of the varnish composition; and
      optionally propylene glycol in an amount of from about 1 to about 5 wt % of the varnish composition;
   iv) a photoinitiator in an amount of from about 0.1 to about 2 wt % of the varnish composition;
   v) a macromolecular emulsifier consisting of polyvinyl pyrolidone (PVP) in an amount of from about 1 to about 10 wt % of the varnish composition; and
b) a pigment composition comprising:
   i) 2-hydroxyethylmethacrylate (HEMA) or salts thereof in an amount of from 1 to 10 wt % of the varnish composition;
   ii) pigment in an amount of from about 0.1 to about 15 wt % of the varnish composition; and
   iii) a filler in an amount of from about 1 to about 20 wt % of the varnish composition.

In a further aspect, the invention provides a dental varnish composition comprising:
a) a resin composition comprising:
   i) an adhesive resin comprising the acidic monomer 10-methacryloyloxydecyl dihydrogen phosphate (MDP) in an amount of from about 10 to about 30 wt % of the varnish composition;
   ii) a base resin comprising the high molecular weight monomer bisphenol A glycidyl methacrylate (Bis-GMA) in an amount of from about 15 to about 30 wt % of the varnish composition;
   iii) a solvent comprising:
      acetone in an amount of from about 15 to about 35 wt % of the varnish composition;
      water in an amount of from about 10 to about 25 wt % of the varnish composition; and
      optionally propylene glycol in an amount of from about 1 to about 5 wt % of the varnish composition;
   iv) a photoinitiator in an amount of from about 0.1 to about 2 wt % of the varnish composition;
   v) a macromolecular emulsifier consisting of polyvinyl pyrolidone (PVP) in an amount of from about 1 to about 10 wt % of the varnish composition; and
b) a pigment composition comprising:
   i) 2-hydroxyethylmethacrylate (HEMA) or salts thereof in an amount of from 1 to 10 wt % of the varnish composition;
   ii) pigment in an amount of from about 0.1 to about 15 wt % of the varnish composition; and
   iii) optionally a filler in an amount of from about 1 to about 20 wt % of the varnish composition.

Each individual component of the dental varnish composition is commercially available, for example:
MDP: Watson International
Bis-GMA: Sigma Aldrich
PVPK90: Ashland
Propylene glycol: Sigma Aldrich
HEMA: Sigma Aldrich
PEGDMA: Esschem
TEGDMA: Sigma Aldrich
TPO: Sigma Aldrich
CQ: Sigma Aldrich
DMAEMA: Sigma Aldrich
DPI: Sigma Aldrich
Mica: KTZ®

The dental varnish composition comprises a resin. The resin comprises a co-monomer mixture of an adhesive resin and a base resin.

On exposure to light the photoinitiator is activated, forming free radicals which initiate the polymerisation and/or copolymerisation of the monomers to form the resin.

The adhesive resin enables the composition to adhere or attach to the tooth enamel surface. The adhesive resin comprises or consists of the acidic monomer 10-methacryloyloxydecyl dihydrogen phosphate (MDP) in an amount of from about 10 to about 30 wt % of the varnish composition. Typically, the MDP is present in an amount of from about 10 to about 25 wt %, optionally about 15 to about 25 wt %, optionally about 18 to about 22 wt %, optionally about 20 wt % of the varnish composition.

### MDP has the Structure:

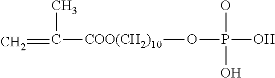

The base resin comprises or consists of the high molecular weight monomer bisphenol A glycidyl methacrylate (Bis-GMA) in an amount of from about 15 to about 30 wt % of the varnish composition. Preferably, the Bis-GMA is present in an amount of from about 15 to about 25 wt %, optionally about 15 to about 20 wt %, optionally about 20 to about 25 wt %, optionally about 16 to about 22 wt %, and optionally about 20 wt % of the varnish composition.

### Bis-GMA has the Following Structure:

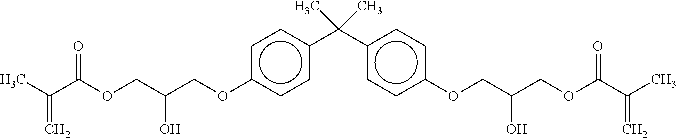

The resin composition comprises a solvent comprising one or more alcohols in an amount of from about 15 to about 35 wt % of the varnish composition; water in an amount of from about to about 25 wt % of the varnish composition; and optionally propylene glycol in an amount of from about 1 to about 5 wt % of the varnish composition. In a preferred embodiment, the propylene glycol is present in an amount of from about 1 to about 5 wt % of the varnish composition.

In preferred embodiments, the one or more alcohols are present in an amount of from about to about 30 wt %, optionally about 15 to about 25 wt %, optionally about 20 to about 25 wt %, optionally about 24 to about 25 wt % of the varnish composition. In a preferred embodiment, the one or more alcohols comprises ethanol, 1-propanol, 2-propanol, 1-butanol, 2-butanol, 2-methyl-1-propanol or combinations thereof. Preferably, the one or more alcohols comprise or consist of ethanol.

Preferably, the water is present in an amount of from about 12.5 to about 22.5 wt %, optionally about 14 to about 21 wt % of the varnish composition.

Preferably, the propylene glycol is present in an amount of from about 2 to about 4 wt %, optionally about 3 wt % of the varnish composition. Propylene glycol is a solvent and levelling agent. Propylene glycol reduces the drying rate of the product and enhances the fluidity of the varnish that dries as a glossy layer. When propylene glycol is present in the dental varnish composition the cured varnish is glossier than when propylene glycol is absent from the dental varnish composition. When propylene glycol is too concentrated in the formula, the varnish remains slightly tacky and softer after cure.

The dental varnish composition comprises a photoinitiator. Photoinitiators used in the present invention are commercially available. The photoinitiator is present in an amount of from about 0.1 to about 2 wt % of the varnish composition.

In one embodiment, the photoinitiator is present in an amount of from about 0.2 to about 1 wt %, optionally about 0.3 to 0.5 wt %, optionally about 0.4 wt % of the varnish composition.

The photoinitiator absorbs light of a particular wavelength. In the present invention a light source having a wavelength of from 380 nm to 550 nm, preferably 400 to 410 nm is used. The light source is provided by a commercially available light curing machine, for example a Bluephase Style curing light from Ivoclar Vivadent Inc.

Typically, the photoinitiator comprises camphoroquinone (CQ) in combination with a co-initiator, trimethylbenzoylphenyl phosphate (TPO), monacylphosphine oxide, bisacylphosphine oxide, or a benzoyl germanium derivative and the composition optionally further comprises a photosensitizer. The benzoyl germanium derivative may be benzoyltrimethylgermane or dibenzoyldiethylgermane. Typically the photosensitizer is diphenyliodonium salts.

In one embodiment the photoinitiator is CQ and the co-initiator is DMAEMA. In one embodiment the photoinitiator is TPO.

These photoiniators are selected because they are not significantly affected by the acidic conditions of the varnish resin.

On exposure to light, photocatalysis of the initiator compound forms radicals which polymerise the monomers. Typically, the methacrylate groups of the monomers MDP and bisGMA react to form crosslinked polymers. The phosphate ester group of the MDP molecule provides chemical adhesion to enamel.

The resin composition further comprises a macromolecular emulsifier consisting of polyvinyl pyrolidone (PVP) in an amount of from about 1 to about 10 wt % of the varnish composition. The PVP acts as a stabilizer, viscosity enhancer and film-forming agent. In the absence of PVP the polar solvent mixtures (e.g., water, alcohols) have a tendency to phase separate from less polar adhesive and base resin combinations. PVP K-90 enhances pigment wetting preventing sediment formation. PVP K-90 also imparts acceptable viscosity to the final product, as well as good handling with brush and on enamel. The PVP-90 also enables self-levelling after the product is applied onto the tooth surface.

Typically, the PVP is present in an amount of from about 3 to 8 wt %, optionally about 4 to about 6 wt % of the varnish composition. In a preferred embodiment, the PVP has weight average molecular weight of at least about 400,000 Da, optionally about 400,000 Da to about 3.5 M Da, optionally wherein the PVP comprises PVP-90K.

PVP polymers have been commercially available in several viscosity grades, ranging from low to high molecular weights. This range, coupled with solubility in aqueous and solvent based systems combined with its nontoxic character, hydrolytic and acidic stability have been some of the properties that has granted PVP polymers great flexibility across multiple applications.

Ashland and BASF offer pharmaceutical and agriculture grades of PVP polymer; PVP K-90 from Ashland has a weight average molecular weight of 1,700,000 g/mol and number average molecular weight of 1,000,000 g/mol. On the other hand, PVP K-30 has a number average molecular weight of 40,000 g/mol and weight average molecular weight of 80,000 g/mol.

The weight average molecular weight of the PVP is determined using Gel Permeation Chromatography using a polymeric standard soluble in water, typically polyethylene glycol or sulfonated polystyrene.

The dental varnish composition further comprises a pigment composition comprising:
i) 2-hydroxyethylmethacrylate (HEMA) or salts thereof in an amount of from 1 to 10 wt % of the varnish composition;
ii) pigment in an amount of from about 0.1 to about 15 wt % of the varnish composition; and
iii) a filler in an amount of from about 1 to about 20 wt % of the varnish composition.

Preferably, the pigment composition is present in an amount of from about 10 to 16 wt %, optionally about 12 wt % of the varnish composition.

Preferably, the HEMA is present in an amount of from about 3 to about 9 wt %, optionally about 4 to about 8 wt % of the varnish composition. HEMA is a pigment dispersion agent and liquid carrier. HEMA is useful as a pigment dispersion agent and liquid carrier while grinding down the particles of solids such as mica, titanium dioxide, cristobalite and iron oxide pigments under high shear when preparing the pigment composition.

### HEMA has the Following Structure:

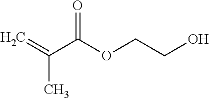

Typically, the pigment comprises one or more of mica, titanium dioxide, cristobalite, barium sulfate, iron oxide and combinations thereof.

Preferably, the filler comprises fumed silica, silica, silicate glass, quartz, barium silicate, strontium silicate, barium borosilicate, borosilicate, lithium silicate, amorphous silica, ammoniated or deammoniated calcium phosphate, alumina, zirconia, tin oxide, or combinations thereof. In one embodiment the filler comprises silicate glass, optionally nanoglass.

In one embodiment the pigment composition comprises HEMA, Mica and/or titanium dioxide and nanoglass.

In one embodiment the pigment composition comprises HEMA and titanium dioxide, wherein the ratio of HEMA to titanium dioxide is from 1:1 to 2:3 in the pigment composition.

In one embodiment the pigment composition comprises HEMA, Mica and nanoglass, where the weight ratio of these components is 8:3:3, respectively in the pigment composition.

In a further embodiment, the pigment composition comprises HEMA, TiO₂ and barium sulfate.

By altering the ratio of the amount of pigment to the remaining components of the composition, different shades of varnish can be prepared. The pigment may be white, black, red, yellow, light brown, or dark brown. In one embodiment a natural tooth colour is preferred. In another embodiment a white tooth colour is preferred. However, a range of colours is contemplated. For example, it may become fashionable to varnish teeth with brightly coloured varnish. The varnish composition may also have a colour suitable to mask colour imperfections on the tooth surface. The varnish composition may be pearlescent, glittery, or textured. The varnish composition may comprise a texturizing agent.

The dental varnish composition may be suitable to mask or cover surface texture imperfections. The dental varnish composition may provide a shiny and/or smooth layer on the tooth surface.

Typically the dental varnish composition is a semi-permanent dental varnish composition. This typically means that the dental varnish composition is capable of being removed and reapplied. The dental varnish composition can be removed by mechanical means, for example by chipping off a layer of dental varnish formed on the teeth. The dental varnish composition can also be removed by applying an appropriate orally-acceptable solvent. The solvent can be applied with a brush, microbrush or sponge.

In some embodiments the adhesive resin additionally comprises the acidic monomer 4-methacryloyloxyethyl trimellitate anhydride (4-META) in an amount of from about 0.5 to about 5 wt %.

### 4-META has the Structure:

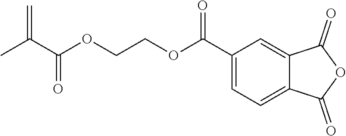 [SMILES: C=C(C)C(=O)OCCOC(=O)c1ccc2c(c1)C(=O)OC2=O]

Typically, the viscosity of the dental varnish compositions should not exceed about 300 cPs and preferably in the range of from about 50 cPs to about 250 cPs.

In a further aspect, the invention provides a cosmetic method of whitening, colouring and/or changing the appearance of teeth, the method comprising applying a dental varnish composition as defined in any preceding claim to the tooth enamel of a subject, and curing the varnish with light having a wavelength in the range of from 380 nm to 550 nm, optionally 400 to 410 nm.

In one embodiment, the tooth enamel is cleaned and dried prior to applying the dental varnish, optionally using dry alcohol and a lint cloth.

In one embodiment of the method, after curing the varnish any excess varnish composition is removed.

In a preferred embodiment of the method, a top coat composition is applied after the curing step, optionally wherein the top coat composition forms a transparent layer.

The top coat composition is typically resistant to aqueous solutions such as saliva, i.e. the composition is not soluble in aqueous solutions. The top coat composition also provides a permanent gloss.

In one embodiment, the top coat composition comprises a resin which is soluble in ethanol, optionally nitrocellulose, cellulose acetate butyrate or polyvinyl butyrate. These resins are commercially available: nitrocellulose (WALSRODER™ NC Standard Grades and Free-Flowing Grades from DuPont/Dow Chemical), cellulose acetate butyrate (CAB 554-04, Eastman Chemical), and polyvinyl butyral (Butvar b-72, Butvar b-76, Butvar b-90, Butvar b-98 from Eastman Chemical) resins.

the top coat composition comprises a an acrylate resin formed by curing one or more monomers comprising from two to five vinyl reactive groups, optionally wherein the top coat comprises triethylene glycol dimethacrylate (TEGDMA), bisphenol A-glycidyl methacrylate (bisGMA), pentaerythritol tri-acrylate, or pentaerythritol tetra-acrylate or combinations thereof.

TEGDMA has the following structure:

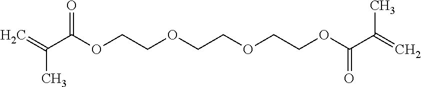 [SMILES: C=C(C)C(=O)OCCOCCOCCOC(=O)C(=C)C]

In one embodiment, the dental varnish composition and/or the top coat composition are applied with a brush, optionally a brush comprising bristles made from a polymer selected from polypropylene, polyethylene, polyamide, or polycarbonate, optionally wherein the brush has a plurality of bristles each having a length of between about 10 to about 15 mm, preferably about 11 to about 14 mm, and a diameter of between about 50 and 75 microns. Each brush may contain between 250 and 400 bristles, preferably 320, that are attached to a plastic stem. The length of stem is preferably between about 25 and 30 mm for ideal handling.

In one embodiment, the method further comprises the step of mechanically removing the varnish.

In a further aspect, provided is a process for preparing a dental varnish wherein:
a) the resin composition components a)i) to v) as defined in claim 1 are mixed;
b) the pigment composition components b)i) to iii) as defined in claim 1 are mixed;
c) then the resin composition mixture formed in step a) and the pigment composition mixture formed in step b) are mixed to provide the dental varnish composition;
wherein mixing of the pigment composition in step b) involves preparing the pigment composition as a paste comprising HEMA, and agitating the composition using a high shear homogenizer that operates at about 200 to about 3,000 rpm minimum rate of shear, a three-roll-mill, or a ball mill system.

The above method enables better dispersion, final coating texture and handling of the inorganic additives like fillers and pigments.

All of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Systeme International de Unites (SI) accepted form.

Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10.

Non-limiting examples will now be described with reference to the following figures:

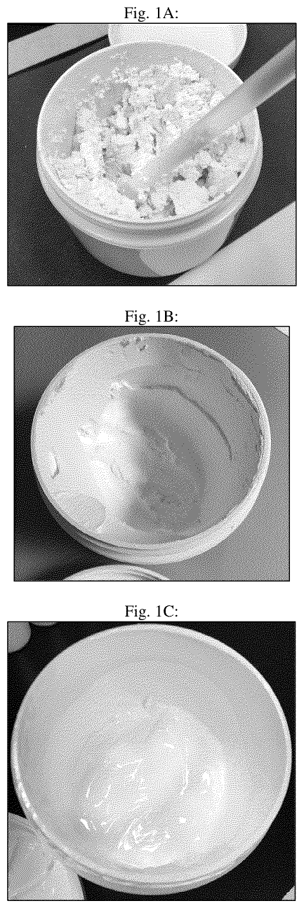

FIG. 1 shows photographs of pigment pre-mix compositions, wherein (A) shows the ground A2 TiO₂/MMA mixture, (B) shows the ground A3 TiO₂/TEGMA mixture, and (C) shows the ground A4 TiO₂/HEMA mixture.

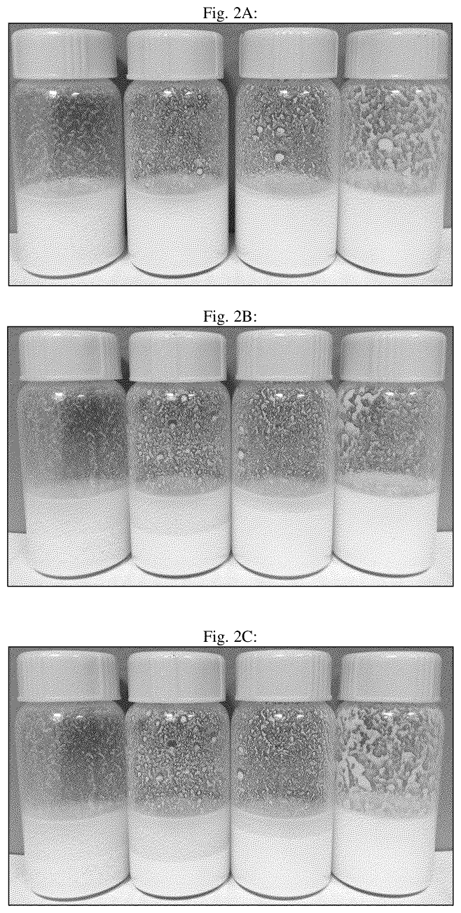

FIG. 2 shows the L3 series of samples as a demonstration of phase separation, wherein (A) shows formulations L3-E, L3-F, L3-G, and L3-H (L-R) after 2 hours and 20 minutes settling, (B) shows formulations L3-E, L3-F, L3-G, and L3-H H (L-R) after 5 hours and 11 minutes settling, and (C) shows formulations L3-E, L3-F, L3-G, and L3-H (L-R) after 6 hours and 31 minutes settling.

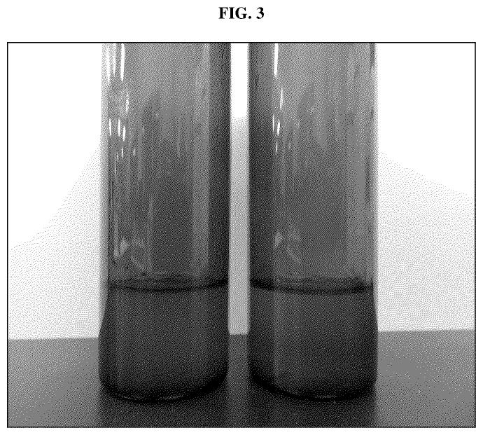

FIG. 3 shows formulations OA-3 and OA-4 (L-R) without HEMA/TiO₂ after 16 hours and 20 minutes of settling as a demonstration of phase separation.

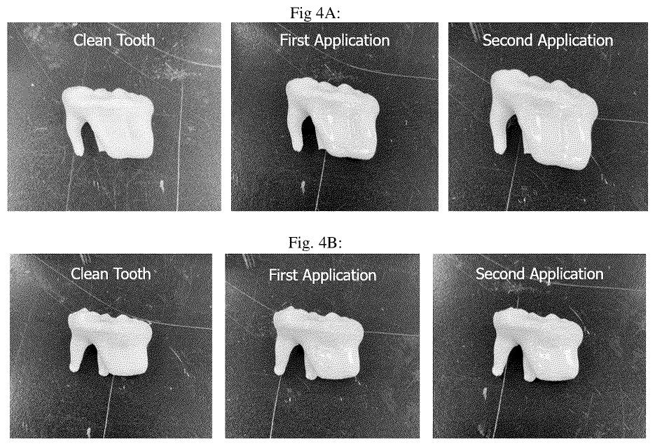

FIG. 4 shows H series samples cured on bear teeth, wherein (A) shows H-1 applied to bear teeth and cured, and (B) shows H-2 applied to bear teeth and cured.

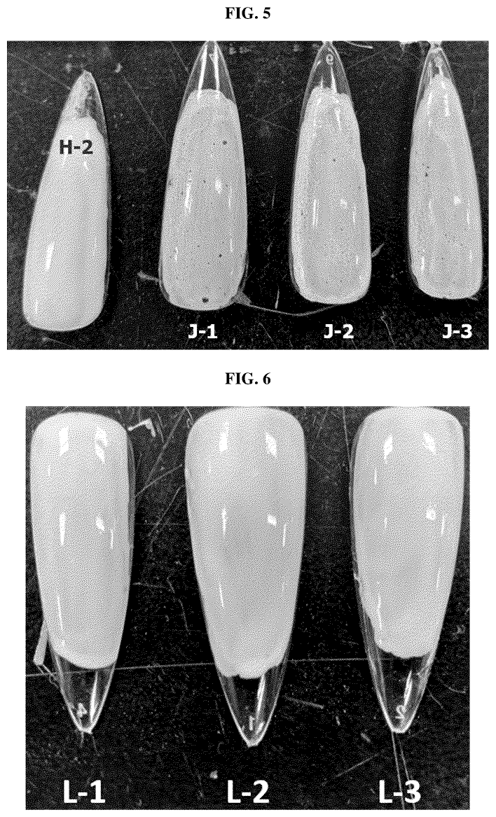

FIG. 5 shows a comparison of H and J series samples cured on polycarbonate substrates.

FIG. 6 shows L series samples cured on polycarbonate substrates.

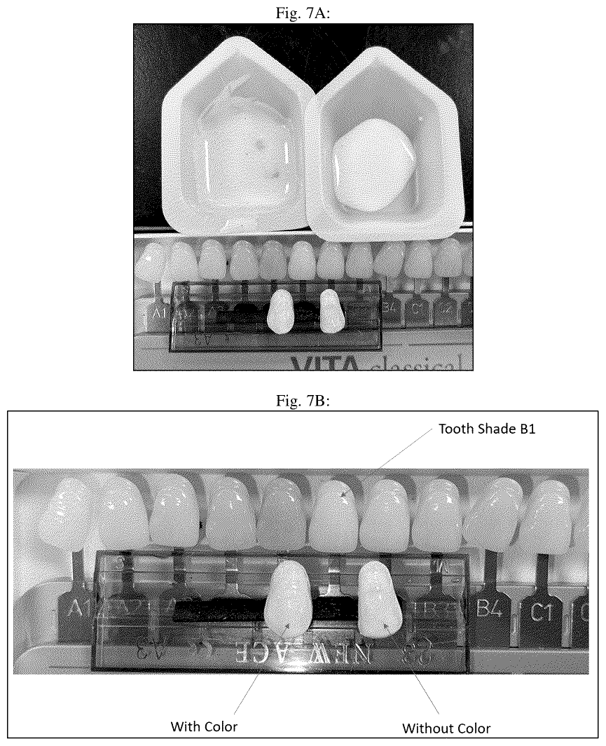

FIG. 7 shows O-3 and O-3-4 pigment composition sample comparison. (A) shows sample O-3-4 with and without color compared to tooth shade B1, and (B) shows an enlargement of sample O-3-4 with and without color compared to tooth shade B1. Compositions with color comprise trace amounts of iron oxide.

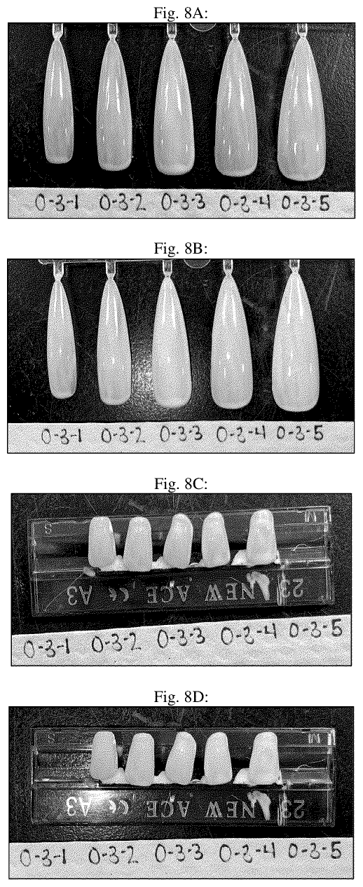

FIG. 8 shows the O-3 series of pigment compositions with BaSO4 samples cured on substrates. (A) shows a single coating on polycarbonate substrate, (B) shows a double coating on polycarbonate substrate, (C) shows a single coating on fake teeth, and (D) shows a double coating on fake teeth.

### EXAMPLES

### Example 1—Pigment Composition Preparation

Pigment pre-mix compositions were prepared using a three-roll mill from Torrey Hills Technologies (T10 model). This model is generally suitable for mixing cosmetics, pharmaceuticals, paints, printing inks, adhesives, epoxy, sealants, and many other pigmented and particle containing mixtures.

Mixtures can also be prepared in a high shear mixer. For example, a 2:3 HEMA/TiO₂ mixture was prepared by combining 120 grams of titanium dioxide and 80 grams of HEMA. This mixture was manually stirred, then homogenized with a Ross high shear mixer at approximately 2000 RPM until a smooth, creamy consistency was achieved.

TABLE 1
Pigment Compositions

| Composition | Components | Ratio (w/w) |
|---|---|---|
| A1 | HEMA:TEGDMA:TiO₂ | 15:1.43:10 |
| A2 | MMA:TiO₂ | 21:19 |
| A3 | TEGDMA:TiO₂ | 21:19 |
| A4 | HEMA:TiO₂ | 1:1 |
| A5 | HEMA:TiO₂ | 2:3 |
| A6 | HEMA:TiO₂:BaSO₄ | 2:2:1 |
| A7 | HEMA:Nanoglass:TiO₂ | 4:3:3 |
| A8 | HEMA:Nanoglass:Mica/TiO₂ | 8:3:3 |

The A2 mixture of MMA and TiO₂ resulted in a clumpy mixture (FIG. 1, A). The A3 mixture of TEGDMA and TiO₂ resulted in a more homogenous and thick mixture (FIG. 1, B). The A4 mixture of HEMA and TiO₂ resulted in a homogenous, glossy mixture (FIG. 1, C). Mixture A5 modifies the HEMA to TiO₂ ratio to give a more uniform paste, with suitable stability for use as a pigment pre-mix composition. Mixture A6, which additionally to HEMA and TiO₂ includes BaSO4 was less opaque and more translucent in appearance. Mixture A6 is also suitable for use as a pigment pre-mix composition. Mixture A7, which additionally to HEMA and TiO₂ includes nanoglass and mica, provides attractive pastel white colouring, with more gloss, good shimmer and translucency. For example, SCHOTT 8235 0.7 μm glass filler functions as an appropriate low Si nanoglass in A7. Other examples of appropriate nanoglasses include SCHOTT 400 nm and 700 nm glass fillers. Mixture A7 is also suitable for use as a pigment pre-mix composition. Mixture A8 modifies mixture A7 by altering the ratio of components and substituting TiO₂ with (8:2 Mica:TiO₂). For example, Natural Shimmer pigment (8:2 Mica:TiO₂) from Making Cosmetics is appropriate. This cosmetic grade additive is analogous to natural mineral TiO₂ with less whiteness imparted to the formula. Mixture A8 is also suitable for use as a pigment pre-mix composition.

### Example 2—Resin Preparation

Compositions are based on weight percentage as described in Table 2 below.

Compositions are mixed in a vortexer with preferred mixing order disclosed in Example 5. The material is applied using a microbrush or similar dental apparatus directly onto lightly surface dried enamel. The material is then agitated over the surface for approximately 30s and lightly air dried to evaporate the solvent. A homogenous coating, no larger than the tip of the light curing unit is recommended. The material is then irradiated for 30s at a minimum of 1000 mW/cm² with either a commercial dual peak LED light source currently available, or a bespoke light source with peak spectral output between 400-410 nm. For example, a Bluephase Style polymerization light can be used. Photocatalysis of the initiator and co-initiator compounds form radicals which co-polymerise functional methacrylate groups of each monomer simultaneously, e.g. crosslinking between the functional groups of bis-GMA and MDP. The dihydrogen phosphate ester group of the MDP molecule provide chemical adhesion to enamel by etching the surface and forming chemical bonds with calcium and hydroxy apatite.

TABLE 2
Resin Compositions

| | Composition (wt %) | | | | | | | | | | | |
|---|---|---|---|---|---|---|---|---|---|---|---|---|
| Material | L3-E* | L3-F* | L3-G* | L3-H* | L3-I* | OA-3 | OA-4* | H-1 | H-2* | H-3 | J-1* | J-2* |
| Water | 16.2 | 16.2 | 16.2 | 16.2 | 16.2 | 16.2 | 16.2 | 20.0 | 20.0 | 21.0 | 22.0 | 21.5 |
| NC | 2.0 | 2.0 | 2.0 | 2.0 | 2.0 | — | 0.5 | — | — | — | — | — |
| HEC | — | — | — | — | — | 1.0 | — | — | — | — | — | — |
| PVP 30K | 12.0 | 12.0 | 12.0 | 12.0 | 12.0 | 12.0 | 12.0 | — | — | — | — | — |
| PVP 90K | — | — | — | — | — | — | — | 4.0 | 4.0 | 4.0 | 4.0 | 4.0 |
| Propylene | — | — | — | — | — | — | — | — | — | — | — | — |
| Glycol | | | | | | | | | | | | |
| 4-META | 20.0 | 4.0 | 4.0 | 4.0 | 4.0 | 4.0 | 4.0 | — | 5.0 | — | 5.0 | 5.0 |
| MDP | 0 | 16.0 | 16.0 | 16.0 | 16.0 | 16.0 | 16.0 | 20.0 | 20.0 | 21.0 | 20.0 | 20.0 |
| Bis-GMA | 11.6 | 11.6 | 7.7 | 15.5 | 15.5 | 18.5 | 19.0 | 20.0 | 20.0 | 21.0 | 20.0 | 20.0 |
| TEGDMA | 11.6 | 11.6 | 15.5 | 7.7 | — | — | — | — | — | — | — | — |
| Ethanol | 16.2 | 16.2 | 16.2 | 16.2 | 16.2 | 16.2 | 16.2 | 20.0 | 20.0 | 21.0 | 22.0 | 21.5 |
| TPO | 0.4 | 0.4 | 0.4 | 0.4 | 0.4 | 0.4 | 0.4 | 0.4 | 0.4 | 0.4 | 0.4 | 0.4 |
| Pigment | 10.0 | 10.0 | 10. | 10.0 | 10.0 | 2.0 | 2.0 | 16.0 | 11.0 | 12.0 | 7.0 | 8.0 |
| pre-mix | A4 | A4 | A4 | A4 | A4 | A4 | A4 | A5 | A5 | A5 | A5 | A5 |
| HEMA | — | — | — | — | 7.7 | 9.7 | 9.7 | — | — | — | — | — |
| Aerosil | — | — | — | — | — | 4.0 | 4.0 | — | — | — | — | — |
| 150 | | | | | | | | | | | | |

| | Composition (wt %) | | | | | | | | | | |
|---|---|---|---|---|---|---|---|---|---|---|---|
| Material | J-3* | L-1 | L-2 | L-3 | N-3 | O-1 | O-2 | O-3 | O-4 | O-3-4 | P-1 |
| Water | 21.0 | 21.0 | 20.0 | 19.0 | 16.25 | 16.2 | 15.7 | 15.2 | 16.3 | 15.2 | 14.1 |
| NC | — | — | — | — | — | — | — | — | — | — | — |
| HEC | — | — | — | — | — | — | — | — | — | — | — |
| PVP 30K | — | — | — | — | — | — | — | — | — | — | — |
| PVP 90K | 4.0 | 4.0 | 4.0 | 4.0 | 4.75 | 4.4 | 4.4 | 4.4 | 4.3 | 4.4 | 5.7 |
| Propylene | — | — | — | — | 2.0 | 2.0 | 2.5 | 3.0 | 2.0 | 3.0 | 3.0 |
| Glycol | | | | | | | | | | | |
| 4-META | 5.0 | — | — | — | — | — | — | — | — | — | — |
| MDP | 20.0 | 20.0 | 20.0 | 20.0 | 20.0 | 20.0 | 20.0 | 20.0 | 20.0 | 20.0 | 20.0 |
| Bis-GMA | 20.0 | 20.0 | 20.0 | 20.0 | 20.0 | 20.0 | 20.0 | 20.0 | 20.0 | 20.0 | 20.0 |
| TEGDMA | — | — | — | — | — | — | — | — | — | — | — |
| Ethanol | 21.0 | 23.0 | 24.0 | 25.0 | 25.0 | 25.0 | 25.0 | 25.0 | 25.0 | 25.0 | 24.8 |
| TPO | 0.4 | 0.4 | 0.4 | 0.4 | 0.4 | 0.4 | 0.4 | 0.4 | 0.4 | 0.4 | 0.4 |
| Pigment | 9.0 | 12.0 | 12.0 | 12.0 | 12.0 | 12.0 | 12.0 | 12.0 | 12.0 | 12.0 | 12.0 |
| pre-mix | A5 | A5 | A5 | A5 | A5 | A5 | A5 | A5 | A5 | A6 | A8 |
| HEMA | — | — | — | — | — | — | — | — | — | — | — |
| Aerosil | — | — | — | — | — | — | — | — | — | — | — |
| 150 | | | | | | | | | | | |

Entries marked * are comparative examples.

The L3 series was prone to separation issues (FIG. 2). Compositions that only included nitrocellulose (NC) or PVP 30K as emulsifiers separated within minutes. The L3 series incorporating both NC and PVP 30K as emulsifiers separated more slowly and to a lesser extent. Across the L3 series the amount of 4-META compared to MDP was adjusted and the amount of TEGDMA compared to Bis-GMA was adjusted. Formulation L3-H was the most stable of L3-E to L3-H, showing minimal separation six hours after mixing. The choice of solvents also played a role in the stability of these example compositions. Acetone-containing mixtures consistently separated much faster than those containing ethanol, indicating that ethanol-water co-solvent systems were far more stable than their acetone-water co-solvent counterparts. Because of this, in preferred embodiments, acetone was completely replaced with ethanol. This also results in improved drying rate. The amount of TEGDMA present also affected the consistency, with higher concentrations of TEGDMA resulting in formulations with low viscosities that separated quickly. In L3-I TEGMA was replaced with additional HEMA, which further reduced separation issues.

The OA series showed that the incorporation of low levels of hydroxyethyl cellulose (HEC), such as in OA-3, resulted in significantly reduced separation compared to even very low levels of NC, such as in OA-4. Even when comprising only 0.5 wt % of the total composition, NC was observed to settle out of the mixture after standing for 16 hours (seen with OA-4), forming a layer of sediment on the bottom of the container (FIG. 3). In contrast, OA-3 standing for 16 hours led to almost no separation at all and only a hint of sediment at the bottom of the container.

The H series performed well, with reasonable levelling and layer thickness on teeth. The liquid was not stringy, and the varnish was not tacky or sticky; the material looked shiny and uniform (see FIGS. 4A and B). The film appeared thin, even after three consecutive applications. 16% levels did not create additional benefit for opacity and cosmetics.

The J series with lower levels of pigment (and higher levels of solvents) were prepared and tested. However, the film quality deteriorated with HEMA:TiO₂ levels below 10% leading to poor coverage on polycarbonate substrates (FIG. 5). The HEMA:TiO₂ pigment paste level should be above 10%.

The L series performed well, providing reasonable coverage after multiple coatings were cured on polycarbonate substrates (FIG. 6). Sample L-3 was characterized to be a better dental varnish than sample, H-2, but L-3 required three consecutive coatings for good surface coverage. However, L-3 maintained reduced gloss following curing. This may be due to higher ethanol content causing faster evaporation, which may result in micro roughness on a coating surface.

Composition N-3 incorporates propylene glycol, which improved drying rate and provided improved self-levelling. This resulted in an improved varnish finish, less prone to cracking, and with more uniform and higher gloss. Glycerin as an alternative did not deliver the same effect.

All O Series samples, with varying levels of propylene glycol and water provided workable varnish samples. O-3 was selected as basis for pigmentation variation.

N and O series compositions are preferred embodiments, with O series compositions particularly preferred.

### Example 3—Pigment Variations

Various pigment mixes with varying levels of HEMA, TiO₂ and BaSO4 were prepared as described above and incorporated at the appropriate level to an O-3 stock formula.

TABLE 3
Pigment Pre-Mix Variations

| | O-3 | O-3-1 | O-3-2 | O-3-3 | O-3-4 | O-3-4 |
|---|---|---|---|---|---|---|
| HEMA (%) | 40 | 40.0 | 40.0 | 40.0 | 40.0 | 40.0 |
| TiO₂ (%) | 60 | 10.0 | 20.0 | 30.0 | 40.0 | 50.0 |
| BaSO₄ (%) | — | 50.0 | 40.0 | 30.0 | 20.0 | 10.0 |

These samples demonstrated that TiO₂ mixed successfully with less opaque pigments to produce more natural white shades (FIG. 7), the strength of which could be varied by multiple applications on polycarbonate or tooth substrates (FIG. 8).

O-3 series compositions are preferred embodiments.

### Example 4—Further Pigment Variations

Various commercially available Mica/TiO₂ powders were ordered from KOBO to incorporate into the P-1 base formula in place of the pigment pre-mix composition. The pigment pre-mix compositions were prepared as described above. These powders had various particle sizes as well as varying ratios of TiO₂ to Mica.

TABLE 4
Pigment Pre-Mix Variations with Various TiO₂/Mica Compositions

| | Composition Ratios (12 wt % total) | | | | | | | |
|---|---|---|---|---|---|---|---|---|
| | P-2 | P-3 | P-4 | P-5 | P-6 | P-7 | P-8 | P-9 |
| HEMA | 8 | 8 | 8 | 8 | 8 | 8 | 8 | 8 |
| SCHOTT 8235 | 3 | 3 | 3 | 3 | 3 | 3 | 3 | 3 |
| Glass Filler | | | | | | | | |
| Classic White | 3 | — | — | — | — | — | — | — |
| Fine White | — | 3 | — | — | — | — | — | — |
| Glam Flake | — | — | 3 | — | — | — | — | — |
| Shimmer White | — | — | — | 3 | — | — | — | — |
| Stellar White | — | — | — | — | 3 | — | — | — |
| Sublime White | — | — | — | — | — | 3 | — | — |
| Superb Silver | — | — | — | — | — | — | 3 | — |
| Ultra Shimmer | — | — | — | — | — | — | — | 3 |

Any of these compositions can be mixed with food, drug and cosmetic grade pigments and lakes, for instance iron oxides or alum lake FD&C Blue 1. The typical concentration of colored dyes are 1-10 unit weight colored pigments per 1,000 unit weight white filler.

P series embodiments are preferred embodiments.

### Example 5—Order of Mixing

Best results were achieved when components were mixed in the order depicted in Table 5, ensuring that that the Part A components of the Batch were fully combined prior to addition of the Part B pigment pre-mix compositions. In mixing of the Part A batch, PVP K-90 is added slowly into water to pre-dissolve the powder and maintain liquid solution form.

TABLE 5
Order of Mixing

| Ingredient | Batch |
|---|---|
| Water | Part A |
| PVP-90K | Part A |
| Propylene Glycol | Part A |
| MDP | Part A |
| Bis-GMA | Part A |
| Ethanol | Part A |
| TPO | Part A |
| Pigment Paste | Part B |

1. A dental varnish composition comprising:
a) a resin composition comprising:
i) an adhesive resin comprising the acidic monomer 10-methacryloyloxydecyl dihydrogen phosphate (MDP) in an amount of from about 10 to about 30 wt % of the varnish composition;
ii) a base resin comprising the high molecular weight monomer bisphenol A glycidyl methacrylate (Bis-GMA) in an amount of from about 15 to about 30 wt % of the varnish composition;
iii) a solvent comprising:
one or more alcohols in an amount of from about 15 to about 35 wt % of the varnish composition;
water in an amount of from about 10 to about 25 wt % of the varnish composition; and
optionally propylene glycol in an amount of from about 1 to about 5 wt % of the varnish composition;
iv) a photoinitiator in an amount of from about 0.1 to about 2 wt % of the varnish composition;
v) a macromolecular emulsifier consisting of polyvinyl pyrolidone (PVP) in an amount of from about 1 to about 10 wt % of the varnish composition; and
b) a pigment composition comprising:
i) 2-hydroxyethylmethacrylate (HEMA) or salts thereof in an amount of from 1 to 10 wt % of the varnish composition;
ii) pigment in an amount of from about 0.1 to about 15 wt % of the varnish composition; and
iii) optionally a filler in an amount of from about 1 to about 20 wt % of the varnish composition.

2. A dental varnish composition comprising:
a) a resin composition comprising:
i) an adhesive resin comprising the acidic monomer 10-methacryloyloxydecyl dihydrogen phosphate (MDP) in an amount of from about 10 to about 30 wt % of the varnish composition;
ii) a base resin comprising the high molecular weight monomer bisphenol A glycidyl methacrylate (Bis-GMA) in an amount of from about 15 to about 30 wt % of the varnish composition;
iii) a solvent comprising:
acetone in an amount of from about 15 to about 35 wt % of the varnish composition;
water in an amount of from about 10 to about 25 wt % of the varnish composition; and
optionally propylene glycol in an amount of from about 1 to about 5 wt % of the varnish composition;
iv) a photoinitiator in an amount of from about 0.1 to about 2 wt % of the varnish composition;
v) a macromolecular emulsifier consisting of polyvinyl pyrolidone (PVP) in an amount of from about 1 to about 10 wt % of the varnish composition; and
b) a pigment composition comprising:
i) 2-hydroxyethylmethacrylate (HEMA) or salts thereof in an amount of from 1 to 10 wt % of the varnish composition;
ii) pigment in an amount of from about 0.1 to about 15 wt % of the varnish composition; and
iii) optionally a filler in an amount of from about 1 to about 20 wt % of the varnish composition.

3. A dental varnish composition of claim 1, wherein the MDP is present in an amount of from about 10 to about 25 wt %, optionally about 15 to about 25 wt %, optionally about 18 to about 22 wt %, optionally about 20 wt % of the varnish composition.

4. A dental varnish composition according to claim 1, wherein the Bis-GMA is present in an amount of from about 15 to about 25 wt %, optionally about 15 to about 20 wt %, optionally about 20 to about 25 wt %, optionally about 16 to about 22 wt %, and optionally about 20 wt % of the varnish composition.

5. A dental varnish composition according to claim 1, wherein the one or more alcohols are present in an amount of from about 15 to about 30 wt %, optionally about 15 to about 25 wt %, optionally about 20 to about 25 wt %, optionally about 24 to about 25 wt % of the varnish composition.

6. A dental varnish composition according to claim 1, wherein the one or more alcohols comprise ethanol, 1-propanol, 2-propanol, 1-butanol, 2-butanol, 2-methyl-1-propanol or combinations thereof.

7. A dental varnish composition according to claim 1, wherein the water is present in an amount of from about 12.5 to about 22.5 wt %, optionally about 14 to about 21 wt % of the varnish composition.

8. A dental varnish composition according to claim 1, wherein the propylene glycol is present in an amount of from about 2 to about 4 wt %, optionally about 3 wt % of the varnish composition.

9. A dental varnish composition according to claim 1, wherein the photoinitiator is present in an amount of from about 0.2 to about 1 wt %, optionally about 0.3 to 0.5 wt %, optionally about 0.4 wt % of the varnish composition.

10. A dental varnish composition according to claim 1, wherein the photoinitiator comprises camphoroquinone (CQ) in combination with a co-initiator, trimethylbenzoylphenyl phosphate (TPO), monacylphosphine oxide, bisacylphosphine oxide, or a benzoyl germanium derivative and the composition optionally further comprises a photosensitizer.

11. A dental varnish composition according to claim 1, wherein the PVP in an amount of from about 3 to 8 wt %, optionally about 4 to about 6 wt % of the varnish composition.

12. A dental varnish composition according to claim 1, wherein the PVP has a weight average molecular weight of at least about 400,000 Da, optionally about 400,000 Da to about 3.5 M Da, optionally wherein the PVP comprises PVP-90K.

13. A dental varnish composition according to claim 1, wherein the pigment composition is present in an amount of from about 10 to 16 wt %, optionally about 12 wt % of the varnish composition.

14. A dental varnish composition according to claim 1, wherein the HEMA is present in an amount of from about 3 to about 9 wt %, optionally about 4 to about 8 wt % of the varnish composition.

15. A dental varnish composition according to claim 1, wherein the pigment comprises mica, titanium dioxide, cristobalite, barium sulfate, iron oxide and any combinations thereof.

16. A dental varnish composition according to claim 1, wherein the filler comprises fumed silica, silica, silicate glass, quartz, barium silicate, strontium silicate, barium borosilicate, borosilicate, lithium silicate, amorphous silica, ammoniated or deammoniated calcium phosphate, alumina, zirconia, tin oxide, or any combinations thereof.

17. A dental varnish composition according to claim 1, wherein the filler comprises silicate glass, optionally nanoglass.

18. A dental varnish composition according to claim 1, wherein the pigment composition comprises HEMA, Mica and/or titanium dioxide, and nanoglass.

19. A dental varnish composition according to claim 1, wherein the pigment composition comprises HEMA and titanium dioxide, wherein the ratio of HEMA to titanium dioxide is from 1:1 to 2:3 in the pigment composition.

20. A dental varnish composition according to claim 1, wherein the pigment composition comprises HEMA, Mica and nanoglass, where the weight ratio of these components is 8:3:3, respectively in the pigment composition.

21. A dental varnish composition according to claim 1, wherein the pigment composition comprises HEMA, TiO₂ and barium sulfate.

22. A dental varnish composition according to claim 1, wherein the adhesive resin additionally comprises the acidic monomer 4-methacryloyloxyethyl trimellitate anhydride (4-META) in an amount of from about 0.5 to about 5 wt %.

23. A cosmetic method of whitening, colouring and/or changing the appearance of teeth, the method comprising applying a dental varnish composition as defined claim 1 to the tooth enamel of a subject, and curing the varnish with light having a wavelength in the range of from 380 nm to 550 nm, optionally 400 to 410 nm.

24. (canceled)

25. (canceled)

26. (canceled)

27. (canceled)

28. (canceled)

29. (canceled)

30. (canceled)

31. A process for preparing a dental varnish wherein:
a) the resin composition components a)i) to v) as defined in claim 1 are mixed;
b) the pigment composition components b)i) to iii) as defined in claim 1 are mixed;
c) then the resin composition mixture formed in step a) and the pigment composition mixture formed in step b) are mixed to provide the dental varnish composition;
wherein mixing of the pigment composition in step b) involves preparing the pigment composition as a paste comprising HEMA, and agitating the composition using a high shear homogenizer that operates at about 200 to about 3,000 rpm minimum rate of shear, a three-roll-mill, or a ball mill system.
